(19) United States
(12) Patent Application Publication
(10) Pub. No.: US 2018/0161478 A1
(43) Pub. Date: Jun. 14, 2018

(54) BARRIER MEMBRANE USED IN PERIODONTITIS TREATMENT AND A PRODUCTION METHOD THEREOF

(71) Applicants: Menemse GUMUSDERELIOGLU, Ankara (TR); Elif SUNAL, Ankara (TR); Tugrul Tolga DEMIRTAS, Ankara (TR); Murat SIMSEK, Ankara (TR)

(72) Inventors: Menemse GUMUSDERELIOGLU, Ankara (TR); Elif SUNAL, Ankara (TR); Tugrul Tolga DEMIRTAS, Ankara (TR); Murat SIMSEK, Ankara (TR)

(21) Appl. No.: 15/574,919

(22) PCT Filed: May 18, 2016
(86) PCT No.: PCT/TR2016/000071

§ 371 Date: Nov. 17, 2017

(30) Foreign Application Priority Data
May 20, 2015 (TR) 2015/06080

(51) Int. Cl. A61L 27/34; A61L 27/58; A61L 27/50; A61L 27/20; A61L 27/56; A61L 27/32; A61L 27/54; A61K 38/18; A61K 31/4164; A61P 1/02; A61M 31/00

(52) U.S. Cl. CPC A61L 27/34; A61M 2210/0631; A61L 27/50; A61L 27/20; A61L 27/56; A61L 27/32; A61L 27/54; A61K 38/1875; A61K 31/4164; A61P 1/02; A61M 31/00; A61L 2300/414; A61L 2300/404; A61L 2420/02; A61L 2400/12; A61M 2207/00; A61L 27/58

(57) ABSTRACT

The present invention relates to a polymeric-based barrier membrane with form memory (1) which is used in periodontitis treatment, comprises, bioactive agents; and a production method (100) of the membrane.

### TECHNICAL FIELD

The present, invention relates to a polymeric-based barrier membrane with form memory which is used in periodontitis treatment, comprises bioactive agents; and a production method of the said membrane.

### BACKGROUND OF THE INVENTION

Periodontitis is a chronic-inflammatory disease which is caused by microorganisms, destroys periodontal tissues and accordingly results in loss of teeth. Purpose of periodontal treatment is not only to stop progressing periodontal disease and also to enable destroyed periodontal tissues to change back into their former states by also realizing periodontal regeneration. Periodontal surgical methods being used nowadays result in repair of periodontal tissues rather than their regeneration.

Methods of Guided Tissue Regeneration (GTR) and Guided Bone Regeneration (GBR) are commonly used in recent years in order to realize periodontal regeneration. In GTR/GBR methods, periodontal regeneration is provided by various membranes developed. Aim of using membrane is to generate barrier between hard tissue and soft tissue. Thereby, migration of epithelial cells to the damaged area is prevented and periodontal regeneration is provided in the damaged area at the same time. Otherwise, epithelial cells migrate to the damaged area and prevent bone regeneration here.

Membranes to be used in GTR/GBR technique need to meet some requirements such as biocompatibility, biodegradability, flexibility and mechanical strength. Aim in these methods is to prevent migration of epithelial cells to damaged area and enable the tooth to recuperate by realizing bone regeneration in the damaged area. For this purpose, various membranes which can resorb or not are used. However, these membranes developed remain incapable in terms of some features required to be included. Among these, there are problems such as: poor mechanical strength, the fact that formation of new bone tissue is not supported sufficiently in the damaged area, rate of resorption is not at desired level, bacterial infection cannot be prevented and epithelial cell migration cannot be avoided. Manipulation difficulties experienced in membrane implantation used is one of the greatest problems of dentists using available commercial products.

The Chinese patent document no. CN102423506, an application in the state of the art, discloses that nanofiber membranes consisting of biodegradable polymer, cellulose nanocrystals and an active medicine are manufactured so as to be used in periodontitis treatment. The invention described in the said document discloses that membrane is manufactured by electrostatic spinning method and there is chitosan in its content.

### SUMMARY OF THE INVENTION

An objective of the present invention is to realize a barrier membrane which provides fast, efficient coating with a composition and characteristics very similar to the hydroxyapatite composition in bone; and a production method of the said barrier membrane.

Another objective of the present invention is to realize a barrier membrane wherein no reduction occurs in dimension of hydroxyapatite particles; and a production method of the said barrier membrane.

Another objective of the present invention is to realize a barrier membrane wherein epithelial cell migration is prevented and inflammation is avoided; and a production method of the said barrier membrane.

Another objective of the present invention is to realize a barrier membrane which is biodegradable, has form memory and osteogenic activity, supports bone regeneration, prevents epithelial cell migration and eliminates inflammation, can be produced easily and inexpensively; and a production method of the said barrier membrane.

### DETAILED DESCRIPTION OF THE INVENTION

“A Barrier Membrane Used in Periodontitis Treatment (1) and a Production Method Thereof (100)” realized to fulfil the objectives of the present invention is shown in the figures attached, in which:

FIG. 1 is a schematic view of the membrane developed by the present invention.

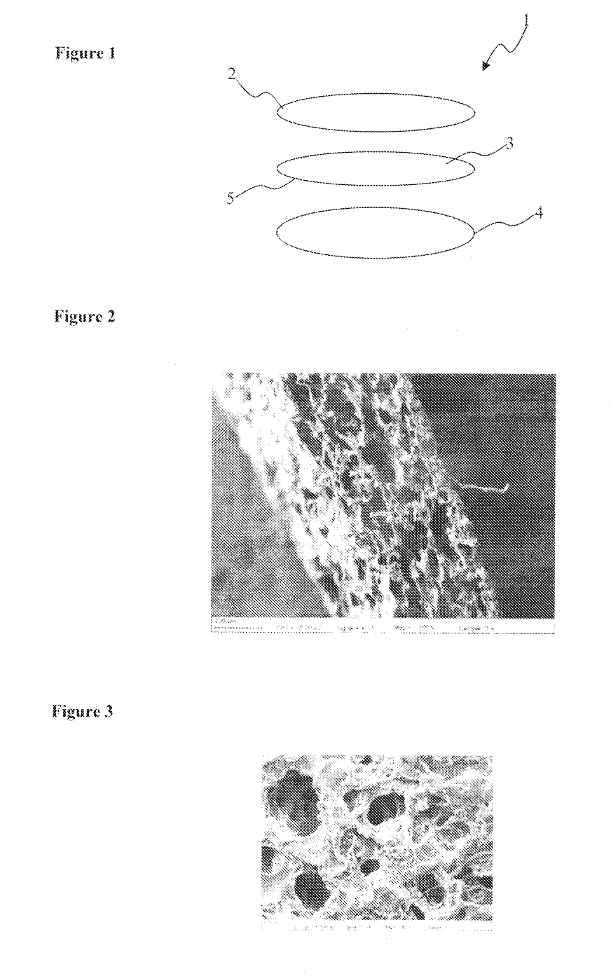

FIG. 2 is a view of the chitosan membrane.

FIG. 3 is a view of the porous surface of the membrane.

FIG. 4 is a view of the porous surface coated with HA.

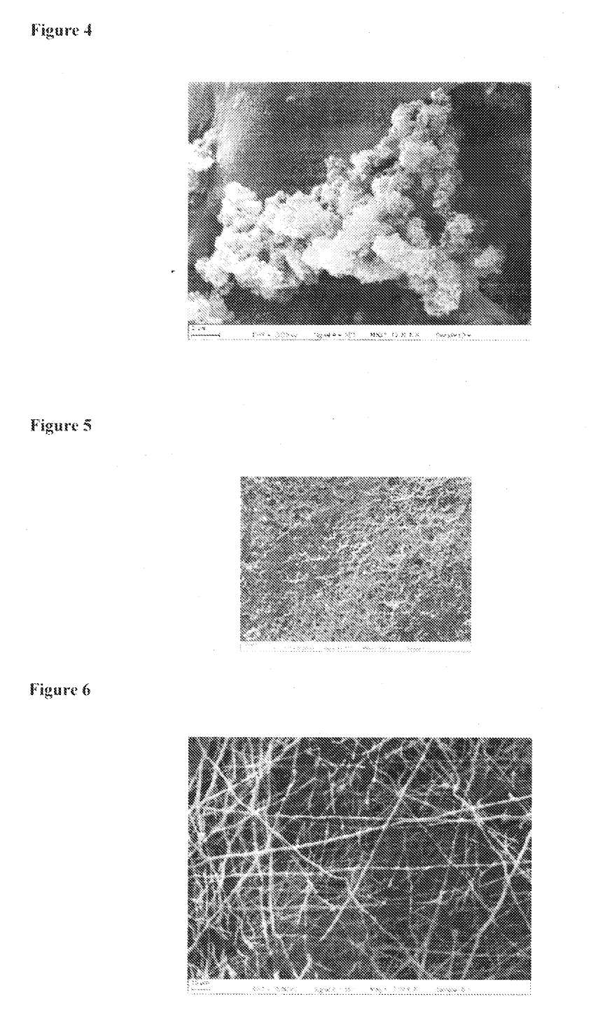

FIG. 5 is a view of the non-porous surface of the membrane.

FIG. 6 is a view of the non-porous surface coated with PCL+Metronidazole.

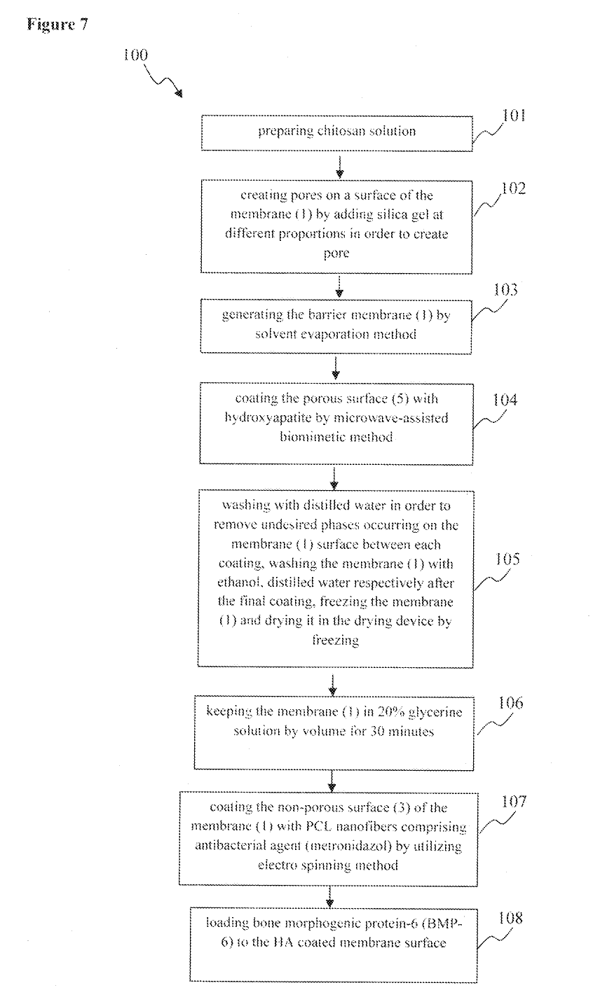

FIG. 7 is a flow diagram of the inventive method.

The components illustrated in the figures are individually numbered, where the numbers refer to the following:

1. Barrier Membrane

2. PCL Nanofiber Coating

3. Non-Porous Surface

4. HA Coating+BMP-6

5. Porous Surface

100. Method

The biodegradable barrier membrane with form memory (1) used in periodontitis treatment, consisting of chitosan, comprises:
at least one non-porous surface (3) which contacts die soft tissue and is coated with PCL (poly-ε-caprolactone) nanofiber coating (2);
at least one porous surface (5) which contacts the hone tissue and is coated with a hydroxyapatite (HA) coating (4) impregnated with bone morphogenetic protein-6 (BMP-6).

The membrane (1) developed by the present invention has chitosan content and it is of form memory and biodegradable nature.

The non-porous surface (3) comprised by the membrane (1) contacts the soft tissue of the tooth where periodontitis treatment will be administered and there is PCL nanofiber coating (2) on thereof.

The porous surface (5) comprised by the membrane (I) is located on the part that will contact the hone tissue and there is a BMP-6 impregnated HA coating (4) on thereof.

In a preferred embodiment of the invention, the non-porous surface (3) is coated with a PCL nanofiber coating (2) comprising an antibacterial agent which is preferably metronidazole.

The inventive method (100) comprises steps of:
preparing chitosan solution (101):
creating pores on a surface of the membrane (1) by adding silica gel at different proportions in order to create pore (102);
generating the barrier membrane (1) by solvent evaporation method (103); coating the porous surface (5) with hydroxyapatite by microwave-assisted biomimetic method (104);
washing with distilled water in order to remove undesired phases occurring on the membrane (1) surface between each coating, washing the membrane (1) with ethanol, distilled water respectively after the final coating, freezing the membrane (1) and drying it in the drying device by freezing ( 105);
keeping the membrane (1) in glycerine solution (106);
coating the non-porous surface (3) of the membrane (1) with PCL nanofibers comprising antibacterial agent by utilizing electro spinning method (107); and
loading bone morphogenic protein-6 (BMP-6) to the HA coated membrane (1) surface (108).

In the inventive method (100), the step of preparing chitosan solution (101) is carried out by dissolving the chitosan—which has preferably 1.1% weight/volume, high molecular weight, deacetylation degree of ≥85%—in acetic acid solution of 1% by volume for 24 hours.

In a preferred embodiment of the invention, the step of coating the non-porous surface (3) of the membrane (1) with PCL nanofibers comprising antibacterial agent by utilizing electro spinning method (107) is carried out by preparing PLC (11% weight/volume, Mn: 70,000-90,000 g/g-mol) upon being mixed within HFIP (Hexafloro-2-propanol) for 24 hours; adding 5%, 10% and 15% (by weight) metronidazole into the PLC structure In order to eliminate the inflammation in the soft tissue; placing the obtained solution into the pump syringe in the electro spinning assembly; and coating the membrane (1) surface with PCL nanofibers for 7 minutes under optimized conditions (107).

In a preferred embodiment of the invention, the method (100) is carried out by adding silica gel—preferably in 150-250 μm diameter—into the solution in order to create pores on a surface of the membrane (1) by adding silica gel at different proportions in order to create pore (102); mixing the solution obtained after adding silica gel preferably for 1 hour and leaving the obtained solution for drying at room temperature (22±2° C.) upon being put into Petri dishes in preferably 5 cm diameter; taking the membrane (1) from the Petri dish and keeping it in 5% NaOH solution (weight/volume) in drying-oven (ES 500, Nüve) of 80° C. for preferably 2 hours and washing it with distilled water at room temperature until a neutral washing solution is obtained. The silica gels are enabled to dissolve inside the solution homogeneously by mixing the solution wherein silica gel is added.

In a preferred embodiment of the invention, the step of coating the porous surface (5) with hydroxyapatite by microwave-assisted biomimetic method (104) is carried out by preparing artificial body fluid solution in order to obtain HA; precipitating the membrane (1) inside 100 ml 10× SBF (Artificial body fluid) solution wherein no NaHCO₃ is added in vacuum drying oven in order to contact all pores with a SBF (Artificial body fluid); transferring it to 100 mL 10× SBF (Artificial body fluid) solution wherein NaHCO₃ is added; and coating the surface with hydroxyapatite by means of microwave at optimized conditions (preferably 600 W, 30 s×9 repeat) (105). HA structure with a composition closest to the bone is obtained under optimized conditions.

Preparing artificial body fluid solution is performed by dissolving the first five salt components given in the Table-1 inside 800 mL distilled water in magnetic stirrer respectively and then completing the volume to 1000 mL.

TABLE 1
Preparation of 10xSBF-like solution

| | Addition | Amount | Concentration |
|---|---|---|---|
| Components | sequence | (g) | (mM) |
| NaCl | 1 | 58.443 | 1000.00 |
| KCl | 2 | 0.373 | 5.00 |
| CaCl₂•2H₂O | 3 | 3.675 | 25.00 |
| MgCl₂•6H₂O | 4 | 1.016 | 5.00 |
| NaH₂PO₄•H₂O | 5 | 0.250 | 3.62 |
| NaHCO₃ | 6 | 0.084 | 10.00 |

In a preferred embodiment of the method (100) realized by the present invention, the glass plate surface in 5×5 cm² dimension is coated with double-sided foam tape for the purpose of coating only the porous surface (5) of the membrane (1) by HA and then the membrane (1) is adhered to the glass plate such that the porous surface (5) of the membrane (1) will be on the upper side and the membrane (1) is attached by plastic clips from its sides in order to prevent the membrane (1) from remove from the surface with liquid medium and the temperature effect.

In an exemplary embodiment of the method (100) realized by the present invention, the step of coating the porous surface (5) with hydroxyapatite by microwave-assisted biomimetic method (104) is carried out by performing coating process for four times by continuing to coat in fresh SBF medium every time in order to obtain sufficient coating on the surface. Washing is performed with distilled water for 1 minute in order to remove undesired phases occurring on the membrane (1) surface between each coating. After the final coating, the membrane (1) is washed with ethanol, distilled water for 10 minutes, 10 minutes respectively. Afterwards, the membrane (1) is kept in −20° C. for one day and dried method (100) comprises the step of keeping the membrane (1) in glycerine solution of 20% (volume/volume) for 30 minutes (106) after the freeze drying process. Following the glycerine treatment (106), the membrane (1) is left for drying at room temperature. Embrittlement of chitosan is eliminated with this application and thereby its manipulability is facilitated both in vitro and in vivo.

In a preferred embodiment of the invention, the step of loading bone morphogenic protein-6 (BMP-6) to the HA coated membrane (1) surface (108) is carried out by impregnation method by utilizing electrostatic interaction between HA and BMP-6 molecules. In a preferred embodiment of the invention, the said step (108) is carried out by dissolving 2.5 μg BMP-6 inside 750 μL PBS (phosphate buffered saline) comprising 2% 0.1 (weight/volume) bovine serum albumin (BSA); taking 30 μL from the obtained solution such that it will contain 100 ng BMP-6; find dripping it to the HA coated membrane (1) surface and keeping the membrane (1) in CO₂ (5%) incubator at 37° C.

In the inventive membrane (1) and method (100), efficiency of BMP-6 in periodontal regeneration is ensured and osteoblastic cells are enabled to support cellular functions upon being loaded to membranes. In addition, porous chitosan supports proliferation and tissue regeneration of the membrane surface (5) consisting of biomimetic HA and BMP-6. With the method (100) developed by the present invention, it is ensured that PCL nanofibers are prevented from removing from the surface at physiological medium conditions and release of antibacterial agent loaded to their structures is provided by enabling them to attach to the membrane surface with solvent interaction by coating the non-porous surface (3) of the chitosan membrane with PCL nanofibers comprising antibacterial agent (metronidazol) by utilizing electro spinning method in order to prevent epithelial cells migration and inflammation.

Within these basic concepts, it is possible to develop a great variety of embodiments of the inventive “A Barrier Membrane Used in Periodontitis Treatment (1) and a Production Method Thereof (100)”; it cannot be limited to the examples disclosed herein and it is essentially according to the claims.

1. A biodegradable barrier membrane with form memory (1) used in periodontitis treatment, consisting of chitosan; comprising:
at least one non-porous surface (3) which contacts the soft tissue and is coated with PCL (poly-ε-caprolactone) nanofiber coating (2);
at least one porous surface (5) which contacts the bone tissue and is coated with a hydroxyapatite (HA) coating (4) impregnated with bone morphogenetic protein-6 (BMP-6).

2. A membrane (1) according to claim 1, characterized by the PLC nanofiber coating (2) which comprises an antibacterial agent that is metronidazole.

3. A method (100) used in periodontitis treatment, enabling to produce a barrier membrane (1) consisting of chitosan, comprising the step of
preparing chitosan solution (101); and characterized by
creating pores on a surface of the membrane (1) by adding silica gel at different proportions in order to create pore (102);
generating the barrier membrane (1) by solvent evaporation method (103);
coating the porous surface (5) with hydroxyapatite by microwave-assisted biomimetic method (104);
washing with distilled water in order to remove undesired phases occurring on the membrane (1) surface between each coating, washing the membrane (1) with ethanol distilled water respectively after the final coating, freezing the membrane (1) and drying it in the drying device by freezing (105);
keeping the membrane (1) in glycerine solution (106);
coating the non-porous surface (3) of the membrane (1) with PCL nanofibers comprising antibacterial agent by utilizing electro spinning method (107); and
loading bone morphogenic protein-6 (BMP-6) to the HA coated membrane (1) surface (108).

4. A method (100) according to claim 3, characterized by the step of preparing chitosan solution (101) which is carried out by dissolving the chitosan—which has preferably 1.1% weight/volume, high molecular weight, deacetylation degree of ≥85%—in acetic acid solution of 1% by volume for 24 hours.

5. A method (100) according to claim 3, characterized by the step of preparing PLC with 11% weight/volume, Mn: 70,000-90,000 g/g-mol upon being mixed within HFIP (Hexafloro-2-propanol) for 24 hours; adding 5%, 10% and 15% metronidazole by weight into the PLC structure in order to eliminate the inflammation in the soft tissue: placing the obtained solution into the pump syringe in the electro spinning assembly; and coating the membrane (1) surface with PCL nanofibers for 7 minutes under optimized conditions (107).

6. A method (100) according to claim 3, characterized by the step of creating pores on a surface of the membrane (1) by adding silica gel at different proportions in order to create pore (102) which is carried out by adding silica gel in 150-250 μm diameter into the solution; mixing the solution obtained after adding silica gel preferably for 1 hour and leaving the obtained solution for drying at room temperature (22±2° C.) upon being put into Petri dishes in preferably 5 cm diameter; taking the membrane (1) from the Petri dish and keeping it in 5% NaOH solution (weight/volume) in drying-oven (ES 500, Nüve) of 80° C. for preferably 2 hours and washing it with distilled water at room temperature until a neutral washing solution is obtained.

7. A method (100) according to claim 3, characterized by the step of preparing artificial body fluid solution in order to obtain HA; precipitating the membrane (1) inside 100 mL 10× SBF solution wherein no NaHCO₃ is added in vacuum drying oven in order to contact all pores with a SBF (Artificial body fluid); transferring it to 100 mL 10× SBF solution wherein NaHCO₃ is added; and coating the porous surface (5) with hydroxyapatite by microwave-assisted biomimetic method by means of microwave at optimized conditions (104).

8. A method (100) according to claim 3, characterized by the step of coating the porous surface (5) with hydroxyapatite by microwave-assisted biomimetic method (104) which is carried out by coating the glass plate surface in 5×5 cm² dimension with double-sided foam tape for the purpose of coating only the porous surface (5) of the membrane (1) by HA and then adhering the membrane (1) to the glass plate such that the porous surface (5) of the membrane (1) will be on the upper side and attaching the membrane (1) by plastic clips from its sides in order to prevent the membrane (1) from remove from the surface with liquid medium and the temperature effect.

9. A method (100) according to claim 3, characterized by the step of loading bone morphogenic protein-6 (BMP-6) to the HA coated membrane (1) surface (108) which is carried out by impregnation method by utilizing electrostatic interaction between HA and BMP-6 molecules.

10. A method (100) according to claim 11, characterized by the step of loading bone morphogenic protein-6 (BMP-6) to the HA coated membrane (1) surface (108) which is carried out by dissolving 2.5 μg BMP-6 inside 750 μL PBS (phosphate buffered saline) comprising 2% 0.1 (weight/volume) bovine serum albumin (BSA); taking 30 μL from the obtained solution such that it will contain 100 ng BMP-6; and dripping it to the HA coated membrane (1) surface and keeping the membrane (1) in CO₂ (5%) incubator at 37° C.
